(19) United States
(12) Patent Application Publication
(10) Pub. No.: US 2010/0303671 A1
(43) Pub. Date: Dec. 2, 2010

(54) HAND CLEANSING/SANITIZING METHOD AND APPARATUS

(76) Inventor: Jerome C. Bertrand, Menlo Park, CA (US)

Correspondence Address: IDEPA INC.;ATTN: JOHANNES SCHNEEBERGER, P.O. BOX 358, BRISBANE, CA, 94005, US

(21) Appl. No.: 12/791,752

(22) Filed: Jun. 1, 2010

Related U.S. Application Data
(60) Provisional application No. 61/182,753, filed on May 31, 2009.

(51) Int. Cl. A61L 2/16

(52) U.S. Cl. 422/29; 422/292; 422/119

(57) ABSTRACT

Systems and methods for cleansing and/or sanitizing using a gaseous media are disclosed. Various hand cleansing and/or sanitizing formulations can be emanated from a system in a discrete packet of gaseous media such as a vortex ring. A hand cleansing and/or sanitizing formulation can be projected to a hand with excellent accuracy and cohesiveness. The gaseous medium can include a permanent gas and/or vapor, and can have a liquid and/or solid in a stable aerosolized form.

### CROSS REFERENCE TO RELATED APPLICATIONS

This application claims priority under 35 U.S.C. §119(e) from U.S. Provisional Patent Application Ser. No. 61/182,753 filed on May 31, 2009, the entire contents of which are hereby incorporated by reference.

### FIELD OF THE INVENTION

The present disclosure relates generally to cleansing and/or sanitizing and, more particularly to apparatus and methods for transporting a hand cleansing and/or sanitizing formulation to hands with gaseous media.

### BACKGROUND

Hand cleansing and/or sanitizing (CS) formulations are useful to remove and/or neutralize unwanted substances and/or microorganisms from hands and/or other members of a body. Furthermore, various formulations can neutralize and/or destroy bacteria, viruses and/or other disease vectors.

Generally, various formulations have been delivered in a liquid form to a user hand. The delivery is often performed with a stream and/or droplets comprising the liquid. The stream and/or droplets can be produced in expelling the liquid from a tube, orifice and/or nozzle. In various applications the formulation is expelled with a pressure differential and/or gravity. The pressure differential is often induced with force on a liquid reservoir boundary and/or a pressure of gas applied to liquid in the reservoir.

Delivery of cleansing and sanitizing formulations has generally been inefficient and/or difficult to control. Liquid formulations emitted from a nozzle and/or other dispenser are generally delivered in a predetermined solid angle. Depending on the relative distance and position of a hand with respect to a fluid source, a large portion of an emitted liquid and/or spray bearing the formulation can bypass a target hand, and/or can be dispensed onto a relatively small portion of the hand. Efficient delivery is particularly problematic when a hand is relatively far from or close to the source of conventional liquid jets, stream, and/or sprays. At close range, a stream and/or jet may reach and/or wet a relatively small portion of the hand. Under these circumstances, one portion of a hand can receive excessive amounts of the CS formulation (e.g. the portion exposed to the jet and/or stream), while an insufficient amount is applied to other areas. Furthermore, the excess fluid reaching a small area can drain off and soil a floor or furniture, or it may be necessary for a user to wipe it with tissue or a towel.

A number of conventional devices convey a dripping dose of formulation to a limited area with the expectation that a user will mechanically redistribute it over remaining portions of a hand (e.g. by rubbing each hand over various areas of the other). Such redistribution can be unreliable and some areas of a hand may not receive sufficient amounts of formulation to be sanitized. It is apparent that delivering suitable amounts of sanitizing formulation in a spray of droplets or a jet of liquid directed toward a hand can be problematic.

Accordingly, there is a need for efficient apparatus and methods that can deliver a predetermined quantity of cleansing and/or sanitizing formulation over widespread areas on a hand. There is also a long felt need for methods and/or devices that can effectively deliver CS formulations and be insensitive to a hand's distance from the source.

### SUMMARY

Systems and methods for hand cleansing and/or sanitizing (CS) using a gaseous medium are disclosed. In various aspects, hand cleansing and/or sanitizing formulations in a gaseous medium are deposited onto a hand. The gaseous medium can include a liquid and/or solid ingredient in a stable aerosolized form.

One aspect of the invention is a system for hand cleansing and/or sanitizing using a cleansing and/or sanitizing ingredient in a first gaseous medium. The system comprises a first launching device operable to emanate a packet of the first gaseous medium whereby the packet is sent through an ambient atmosphere to a hand. The system also comprises a first source operable to provide the first cleansing and/or sanitizing ingredient to the first gaseous medium, and at least one fluid passage operable to convey the first gaseous medium to the first launching device. There is an embodiment of the system comprising a plurality of launching devices. In this embodiment, each launching device is operable to send a predetermined quantity of a respective gaseous medium comprising at least one respective cleansing and/or sanitizing ingredient through the ambient atmosphere to the hand. A related embodiment has a launching device operable to send packets of gaseous media in a first direction, and further launching devices operable to send packets of gaseous media in at least another direction. In another embodiment, the first source has the first cleansing and/or sanitizing ingredient in a solid and/or liquid phase and the system further comprises at least one device operable to aerosolize the solid and/or liquid first cleansing and/or sanitizing ingredient.

Another embodiment of the system comprises a chamber operable to confine the first gaseous medium, at least one fluid passage operable to convey the first cleansing and/or sanitizing ingredient from the first source to the chamber, and a pressurizing device operable to provide pressure energy to gaseous media in the launching device. The first launching device is thereby operable to emanate a predetermined quantity of the first gaseous medium that can travel through the ambient atmosphere to the hand. There is an embodiment where the launching device is operable to emanate a vortex ring packet and/or a spherical vortex packet and the emanation comprises imparting an impulse to a portion of gaseous medium, thereby displacing the gaseous medium portion through an opening to the ambient atmosphere. A further embodiment has a launching device operable to send a packet of the gaseous medium in a selectable direction.

A still further embodiment of the system includes a treatment space partially enclosed with partition walls and an exhausted aperture operable to maintain a net airflow from a surrounding space into the treatment space. Another embodiment has a control circuit and a plurality of ingredient sources. The control circuit is operable to select ingredients from among the plurality of sources. It is also operable to inject the selected ingredients into the chamber and to detect a position of the hand. Furthermore, the control circuit is operable to direct the launching device to send a gaseous media packet to the position of the hand.

Another embodiment of the system has a second source operable to provide an indicator ingredient to a gaseous medium. It also has a launching device operable to send a packet of the gaseous medium comprising the indicator ingredient through an ambient atmosphere to the hand. The indicator ingredient is operable to disclose a sufficiency of cleansing and/or sanitizing the hand.

Another aspect of the invention is a method of cleansing and/or sanitizing an object. The method comprises dispensing a cleansing and/or sanitizing ingredient from a first source, directing the cleansing and/or sanitizing ingredient through a fluid passage to a launching device; and emanating a first preselected amount of the gaseous medium comprising the cleansing and/or sanitizing ingredient with the launching device. The emanated gaseous medium is operable to travel from the launching device to an object, and to deposit the cleansing and/or sanitizing ingredient on the object.

In an embodiment of the method, the emanated gaseous medium is in a vortex ring packet. In another embodiment, the object is selected from the group consisting of a living body portion, a hand, and a foot. There is a further embodiment where the cleansing and/or sanitizing ingredient is substantially applied over an area of a hand. Another embodiment of the method comprises sending a packet of gaseous media comprising an indicator ingredient through an ambient atmosphere to the object. The indicator ingredient is deposited on the object, and a degree of cleansing and/or sanitizing of the object is sensed with the indicator ingredient.

There is also an embodiment where a sequence of gaseous media packets having a plurality of distinct compositions is emanated. The emanation is performed in a manner operable to deliver each respective packet composition to the object at a relatively different sequential time. At least each of two distinct compositions have at least one ingredient exclusive from the other. There is a related embodiment, where the two distinct compositions have synergistic effects.

In another aspect of the invention, there is a device that is operable to perform the method of cleansing and/or sanitizing a hand. A still further aspect of the invention is an apparatus comprising means for providing a gaseous medium including a cleansing and/or sanitizing ingredient in a stable aerosol. This aspect also includes means for sending a preselected amount of the cleansing/sanitizing ingredient through an ambient atmosphere to an object. There is an embodiment wherein the preselected amount of the cleansing/sanitizing ingredient is emanated from a launching device in a vortex ring packet. Another embodiment includes means for sending the vortex ring packet in a selectable direction.

### BRIEF DESCRIPTION OF DRAWINGS

Various embodiments are illustrated in an exemplary manner by the accompanying drawings. The drawings and accompanying description should be understood to explain principles of the embodiments rather than be limiting. Other embodiments will become apparent from the description and the drawings.

FIG. 1A is a simplified diagram showing aspects of one apparatus for delivering cleansing and/or sanitizing ingredients.

FIG. 2A is a simplified diagram showing aspects of another apparatus for delivering cleansing and/or sanitizing ingredients.

FIG. 2B is a simplified diagram showing a portion of a cleansing and/or sanitizing apparatus having a chamber with an electromagnetically deflectable wall portion.

FIG. 2C is a simplified diagram showing a portion of a cleansing and/or sanitizing apparatus having a chamber with piston pumping means.

FIG. 3 is a simplified diagram showing aspects of another apparatus for delivering cleansing and/or sanitizing ingredients.

FIG. 4A shows aspects of a piston launcher for delivering cleansing and/or sanitizing ingredients.

FIG. 4B shows aspects of a diaphragm launcher for delivering cleansing and/or sanitizing ingredients.

FIG. 4C shows aspects of a gas displacement launcher for delivering cleansing and/or sanitizing ingredients.

FIG. 5 shows a simplified diagram showing portions of a system for delivering cleansing and/or sanitizing ingredients.

FIG. 6A shows a simplified diagram showing portions of a system for delivering cleansing and/or sanitizing ingredients.

FIG. 6B shows a simplified diagram showing portions of a system for delivering cleansing and/or sanitizing ingredients.

FIG. 7A shows a wall mounted hand cleansing and/or sanitizing system.

FIG. 7B shows a hand cleansing and/or sanitizing system with a floor stand.

FIG. 8 shows a handheld cleansing and/or sanitizing device.

### DETAILED DESCRIPTION

Systems and methods for hand cleansing and/or sanitizing (CS) using a gaseous medium are disclosed. In various aspects, hand cleansing and/or sanitizing formulations in a gaseous medium are deposited onto a hand. The gaseous medium can include a liquid and/or solid ingredient in a stable aerosolized form. Furthermore, the gaseous medium and/or stable aerosol therein can comprise a CS ingredient that is a vapor and/or an ingredient that is a permanent gas, and can also include further synergistic, coactive and/or other CS ingredients such as a fragrance, an indicator ingredient, a colorant, a medicament, and/or further substances useful for the application.

In various aspects, systems for delivering preselected amounts of a gaseous medium having at least one CS ingredient are disclosed. Gaseous media can be useful to transport at least a portion of a liquid and/or solid CS formulation to a hand. Media can deliver various ingredients simultaneously and/or sequentially. For example, there are system embodiments operable to emit and/or deliver a plurality of gaseous media compositions in a predetermined sequence. In some embodiments, different compositions can be directed to a hand from a plurality of sources. The sources can be in different spatial positions. Furthermore, various different compositions can be used cooperatively to effectuate a process for CS.

In one embodiment, a pressurizing device and a launching device can emanate a discrete packet of the gaseous medium from at least one opening of the system. In some embodiments, the opening can be an orifice on a wall of a chamber. However there are embodiments where a packet can be launched from an open tube, an open end of a conical frustrum, and/or other emitting structure (emitter) and having different opening configurations. It has been found that manipulating a dimension or shape of a launching device during emanation can be useful to control a size, velocity, and/or range of an emitted packet of gaseous media. In a number of embodiments, the cross section of a launching device exit opening is static. However, in some embodiments the shape, cross section, or diameter of the exit opening from which a packet emerges can be manipulated during the emanation. For example, there are embodiments in which a packet in the form of a vortex ring is emitted from a variable diameter sloped nozzle. In some of these embodiments, the range and/or velocity of an emergent vortex ring packet can be increased by temporally increasing the exit diameter of a sloped nozzle during vortex ring formation (emanation). In still further embodiments, packets having one or more hand cleansing and/or sanitizing formulations can be sent to a hand from a plurality of launching devices, and/or various packets can be emanated from one or more launching devices during different and/or sequential intervals of time.

An emergent packet of the gaseous medium can travel quite long distances through ambient air while maintaining a relatively stable size and velocity. Hand cleansing and/or sanitizing formulations can thereby be projected to a hand with excellent accuracy and cohesiveness. There can be relatively little expansion and relatively little entrainment of ambient air during packet travel. When such a moving packet of gaseous medium intercepts a hand, there can be a brief swirling wind as the fluidic motion of the packet dissipates. The swirling can distribute a cleansing ingredient in the parcel over a hand, sufficiently reaching various areas and crevices on the front and back of a hand, not in a direct line of sight from the launching device. Thereby, the cleansing and/or sanitizing ingredient can be substantially applied over the area of a hand.

In a preferred embodiment, a hand sanitizing formulation can be aerosolized and injected into the chamber of a system comprising a vortex ring generator. A preselected amount of gaseous medium in the chamber can be launched from an opening in a manner that transforms kinetic and/or pressure energy of a gaseous medium fluid within the system into an emergent free vortex ring. The fluidic motion of a vortex ring can convey a parcel of the gaseous medium along a relatively straight path, through ambient air, to a distal hand. A vortex ring launching device can be configured to launch a vortex ring packet having a predetermined radius and speed. In various embodiments, the content and approximate width of the vortex ring can be generally preserved as it travels to a hand.

Although the system and method are disclosed in terms of examples relating to cleansing and/or sanitizing hands, the systems, methods, and various embodiments can be useful for cleansing and/or sanitizing various other body parts such as feet, arms, and/or other body members of humans and/or animals. Furthermore there are embodiments that can be useful to treat inanimate objects. For example, a system can be configured to emanate vortex rings and/or other forms of gaseous media comprising gaseous cleansing and/or sanitizing compositions, as disclosed herein, to clean and/or sanitize various areas such as a wall portion, a countertop, a switch, a handle, a tool, a utensil, and/or other surfaces.

The terminology herein is for the purpose of describing particular embodiments and is not intended to be limiting. It will be understood that, although the terms first, second, etc. may be used to describe various elements, these terms are only used to distinguish one element from another and the elements should not be limited by these terms. For example, a first element could be termed a second element, and similarly a second element could be termed a first element, without departing from the scope of the instant description. As used herein, the singular forms “a”, “an” and “the” are intended to include the plural forms as well, unless the context clearly indicates otherwise. It will be further understood that the terms “comprises,” “comprising,” “includes,” “including,” and/or “having,” as used herein, are open-ended terms of art that signify the presence of stated features, integers, steps, operations, elements, and/or components, but do not preclude the presence or addition of one or more other features, integers, steps, operations, elements, components, and/or groups thereof. Reference in the specification to “one embodiment”, “an embodiment”, or “some embodiment”, etc. means that a particular feature, structure, or characteristic described in connection with the embodiment is included in at least one embodiment. The appearances of the phrase “in one embodiment” in various places in the specification are not necessarily all referring to the same embodiment, nor are separate or alternative embodiments mutually exclusive of other embodiments.

The term module refers to a distinct unit that is operable to perform an identifiable function. A module can be a self-contained physical unit or piece of equipment. A module can also be a logical component effectuated by a processor and tangible media having instructions and/or data that are operable for the processor to perform the identifiable function. The term automatic refers to a module, service, or control system that is operable to perform a function with no human interaction.

The terms gaseous medium and gas phase species as used herein refer to a mixture of various species that are not bound to each other, have relative thermal motion with respect to each other, and can uniformly fill a container in which they are held. This term includes combinations of species in an aeriform gas mixture, vapor, stable aerosols, and gaseous fluid mixtures thereof.

As used herein, a stable aerosol refers to a suspension of particles and/or droplets (collectively “particles”) in a gas that do not separate by gravity in periods of minutes to hours or more. Typically, particles having a maximum size less than some tens of micrometers to 100 micrometers in diameter can be suspended as a stable aerosol, although the maximum size can depend on temperature, density of the particles, and fluid motion. Dispersions comprising substantial amounts of relatively large and/or heavy droplets and/or particles in a gas that are susceptible to gravitational settling in minutes are not considered stable aerosols. As used herein, aerosolization will be understood to reference production of a stable aerosol.

The term cleansing sanitizing ingredient (CS ingredient) as used herein is understood to include various ingredients useful, in single or in combination, to effectuate a cleansing and/or sanitizing process. Depending on the context, a CS ingredient can be a pure chemical compound or combination of different chemical species operable to clean and/or sanitize, and/or can comprise a synergistic and/or coactive ingredient, and/or a further substance useful for the application such as a fragrance, an indicator ingredient, a colorant, a medicament, and/or other substances useful in a cleansing/sanitizing process and/or application.

Various terms denoting spatial position such as above, below, upper, lower, leftmost, rightmost and the like are to be understood in a relative sense. The various aspects of the apparatuses described herein are operable without regard to the spatial orientation of the apparatuses as a whole. For example, an apparatus can be configured in a vertical orientation or in a horizontal orientation. Hence a component or module that is described as being above another component or module in a first embodiment having a first orientation, could equivalently be described as being to the left of the other component or module in an equivalent second embodiment configured to be in a second orientation 90 degrees counterclockwise with respect to the first.

The present teachings may be embodied in various different forms. In the following description, for purposes of explanation, numerous specific details are set forth in the description and drawings in order to provide a thorough understanding of the various principles. Furthermore, in various instances, structures and devices are described and/or drawn in simplified form in order to avoid obscuring the concepts. However, it will be apparent to one skilled in the art that the principles can be practiced in various different forms without these specific details. Hence aspects of the disclosure should not be construed as being limited to the embodiments set forth herein.

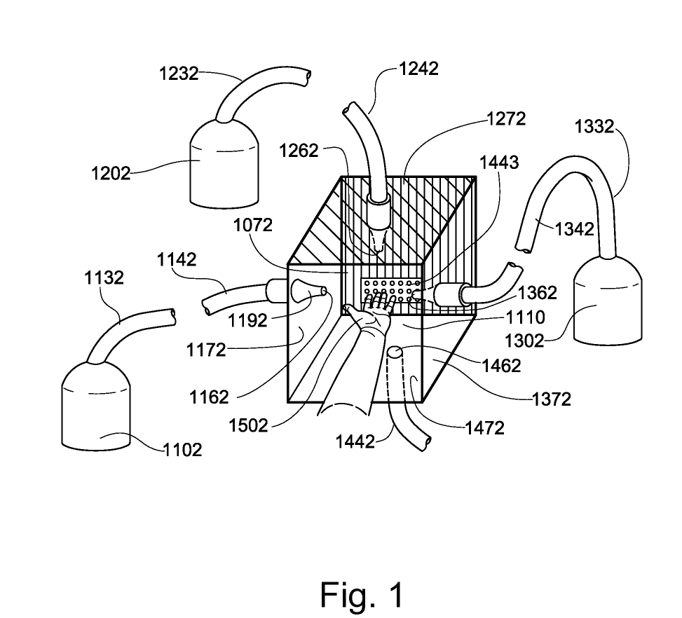

Some embodiments of a CS system delivery system can be understood with respect to the simplified diagram of FIG. 1. A system with respect to FIG. 1 has at least one source 1102 that can provide an CS ingredient. The ingredient can be delivered from the source in a gaseous medium, and/or or it can be transformed into or assimilated in a gaseous medium outside of the source. The source 1102 can deliver a predetermined quantity of the ingredient by way of a fluid passage 1132-1142. The fluid passage can convey a gaseous medium having the ingredient to a launching device 1192. Gaseous medium conveying the CS ingredient can be dispensed from a launching device having an orifice 1162. The launching device can emanate gaseous medium toward a hand 1502, and/or can direct the flow generally into a space 1110 for treating a hand. In a number of embodiments a treatment space can be at least partially enclosed and can be open to an adjacent space. For example, in an embodiment with respect to FIG. 1, a treatment space 1110 can be partially enclosed on 5 sides with partition walls such as 1172, 1272, 1372, 1472. In various embodiments at least some partitions can be solid. In some embodiments one or more partition walls can be perforated and/or can comprise gridded areas or the like. Gridded, perforated, and/or other forms of permeable walls can allow some gas to flow inward and/or outward through to surrounding space, depending on the application. Furthermore, various gaseous media sources can deliver the same and/or different CS ingredients, in single or in combination. For example, different gaseous media sources such as 1102, 1202 and/or 1302 with respect to FIG. 1 can dispense at least some respectively different ingredients through associated fluid passages 1132-1142, 1232-1242, 1332-1342 and launching devices 1292 and/or 1392 into the space 1110, or they can dispense the same ingredient(s). Gaseous media emanating from respective orifices 1162, 1262 and 1362 can be sufficiently confined within the interior space defined by the partitions 1072, 1172, 1272, 1372, and/or 1472. In some embodiments, excess gaseous media can be evacuated through an opening 1462 and line 1442. Some further embodiments provide an exhaust aperture 1443 that can be sealably coupled to an environmental exhaust system. In some embodiments, there is net inward airflow from the surrounding space to the interior 1110 maintained with the partitions and airflow into the exhaust aperture.

A fluid passage such as 1132-1142, 1232-1242, and/or 1332-1342 can be comprised of tubing, ducting, pipe, micromachined channels within a solid body, and/or various other means operable to direct a fluid from a source to a launching device. A launching device can include a conical nozzle such as 1192 coupled to a fluid passage and can comprise an orifice 1162 such as shown with respect to FIG. 1. The launching device can be supported on a wall, but there are further embodiments having a freestanding launching device (e.g. a launching device that is not coupled to a fluid passage in a wall and/or not supported by a wall).

Various launching devices are useful, depending on the embodiment. For example, a launching device can include a simple open jet, a cone nozzle, a convergent and/or convergent divergent nozzle, a swirl nozzle, an eye-ball jet or eye-ball nozzle, a showerhead, a vortex ring launcher, and others. However other launching devices can be used to emit and/or direct the gaseous media to a hand for CS. Furthermore there can be an aimable launching device. For example, a jet can be configured to emanate gaseous media in a predetermined direction and/or in a predetermined conical volume defined by an angle and apex position at a launching device opening. Some embodiments include a hand sensor that can detect the presence and/or position of a hand. Some of these embodiments include a control circuit that can direct the flow of media from a launching device to a hand, responsive to detecting the hand position. Furthermore, a launching device can have means to select a conical divergence angle for an emanated gaseous media. Media spreading transverse to a path to the distal hand can be selectively controlled to have gaseous medium approach a predetermined portion of a hand. For example, in some embodiments the direction and/or divergence of a gaseous packet emanated from an orifice or nozzle can be automatically adjusted based on sensing the position of a hand.

A number of embodiments comprise a wall having fluid passages operable to direct gaseous media to a plurality of orifices and/or nozzles in various positions (collectively referred to as orifices). The orifices can be in suitable positions and can have orientations that are collectively operable to gaseous media over various areas of a hand. For example, one embodiment includes a plurality of orifices, each orifice being associated with a relatively straight section of channel leading to an opening (hole) in the wall. The axis of the channel section can have preselected azimuthal and elevation angles with respect to the wall to select a direction of emergent flow.

There are embodiments with respect to FIG. 1 having at least some conical nozzles protruding from a wall. In some embodiments, gaseous media can be emitted from each of the various nozzles/orifices in respectively predetermined directions. Some embodiments comprise means to adjust the launching direction and/or other attributes of emission gaseous media emanation. There are embodiments having a nozzle, a jet, a vortex ring launcher, and/or other launching means operable to direct fluid toward a target. Various embodiments include a processor and tangible media having stored instructions and data. The processor can perform the instructions and operate on the data to effectuate a module operable to control various aspects of a CS system. For example, there can be a module to select a CS composition, a module to aim a fluid launcher at a hand, a module to effectuate an emission velocity and/or divergence, and other modules. In various embodiments a processor can sense the position of a switch, and/or can receive data from sensors operable to detect hand presence, hand position, a pressure in the CS system, a fill level in an CS source, a chemical composition, a microorganism, an indicator compound on a hand, and/or other things, depending on the application. A time, duration, composition, direction, and/or other result-effective parameters for sending ingredients in a gaseous medium to a target (such as a hand) can be based on information from the sensors. Furthermore, values of such result-effective parameters can be selected with a control circuit.

The flexibility of the disclosed apparatus can support advanced cleansing and/or sanitizing methods that depend on sequentially applying different ingredients to an object in a serial treatment. In some embodiments ingredients that would be antagonistic, unstable, or otherwise incompatible where admixed in a gaseous medium before transport and/or delivery, are sent to an object in different gaseous medium packets so as to transport the mutually incompatible ingredients separately. Various packets having distinct compositions and/or ingredients can be emanated from different launching devices and/or from one launching device in a sequence. Furthermore, depending on the application, packets of gaseous media can be emanated at preselected relative times and/or with different velocities, in a manner operable to deliver their payload ingredients to an object at relatively different sequential times. A time sequence of exposures to different compositions can provide enhanced microbial lethality. Furthermore, in some applications, a first ingredient operable to sterilize (e.g. “kill”) a first group of microorganisms can have relatively little effectiveness against a second group. Similarly, a second ingredient may be effective against second group of microorganisms while being incompatible with gaseous media bearing the first ingredient. For example, although ingredients such as ozone, hydrogen peroxide, peroxyacetic acid, and cyclic antibiotics can be problematic when mixed with one another (e.g. suffer decomposition, reduction in potency, or other unwanted effects), they can be usefully applied in sequence. A time sequenced application of gaseous media separately bearing different ingredients also can have reduced side effects, such as less toxicity for non-target species, in comparison to treatment with gaseous media bearing a mixture of the different ingredients. It can be seen that time sequenced exposures to various compositions can have synergistic effects.

Depending on the applications, a system can have one or more first launching devices configured to emanate gaseous media selected from among a first group of compositions, and one or more second launching devices configured to emanate gaseous media selected from among a second group of compositions. In some of these embodiments at least some compositions selected from different groups can be mutually incompatible. Also, a launching device useful for one group of compositions can have materials of construction that are incompatible with another group.

There are also embodiments, where distinct compositions that would be incompatible and/or unsuitable to emanate in the form of a mixture, or using a common launching device, can be emanated from different launchers at approximately the same time. A number of compositions that would be mutually antagonistic and/or unstable etc. in proximity during prerelease interactions can, nevertheless, be effective during a short period when they are mutually deposited on a hand.

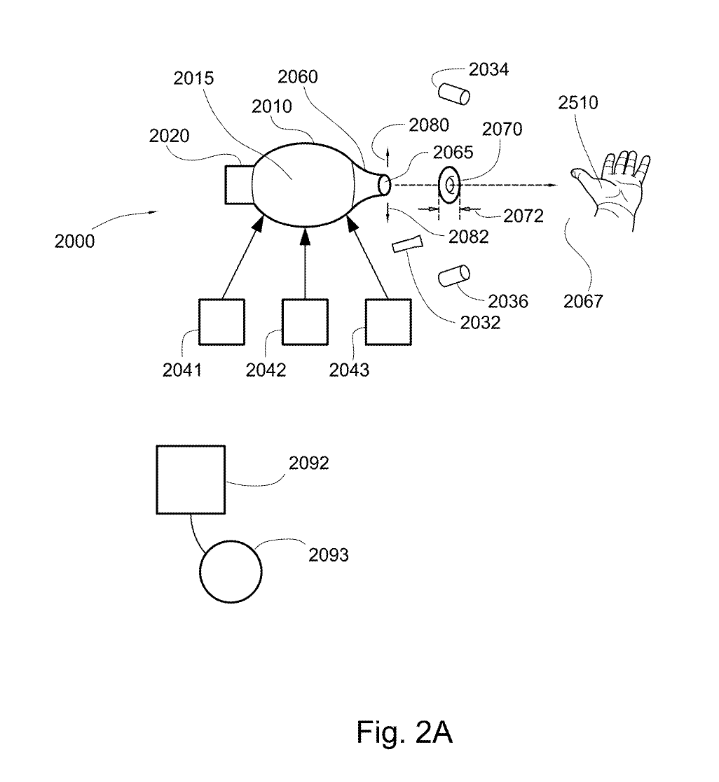

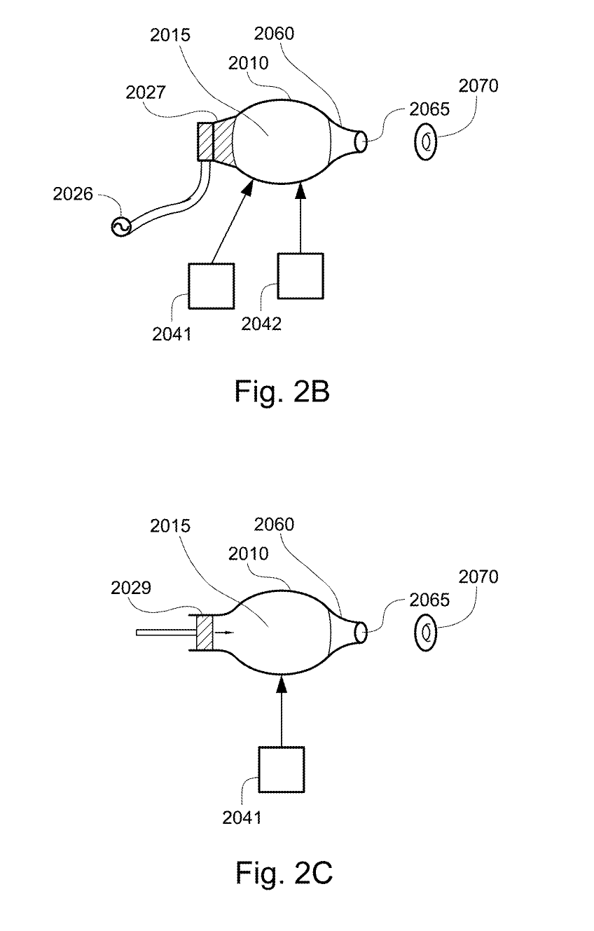

Another hand cleansing and/or sanitizing (CS) system according to this disclosure can be understood with reference to FIG. 2A. A hand cleansing/sanitizing delivery module 2000 can include a chamber 2010, a pressurizing device 2020 to pressurize the inside of the chamber, at least one source 2041 having an CS ingredient, a launcher 2060, and an aperture 2065. The source 2041 is to store and deliver at least one ingredient of an CS formulation into gaseous medium 2015 in the chamber. The ingredient can be in the source 2041 in a liquid, solid, and/or gaseous form. However, in various aspects, a liquid and/or solid ingredient in a source 2041 can be transformed into a gaseous medium form during injection into the chamber 2010. The gaseous form can be a permanent gas, a vapor, and/or a stable aerosol. In some embodiments a liquid and/or a solid ingredient in a source 2041 is aerosolized as it is dispensed into the chamber 2010. For example, a sanitizing ingredient such as a liquid phase alcohol in a source 2041 can be aerosolized into chamber 2010 in a stable form.

In some embodiments a solid and/or liquid ingredient from a source 2041 can be vaporized or sublimated into a gaseous phase form. The vaporization and/or sublimation can be performed before it enters the chamber 2010 and/or can be performed in the chamber, and the ingredient from the source can be admixed with any gaseous media already in the chamber. For example, in some embodiments, a volatile liquid sanitizing and/or cleansing ingredient such as ethanol can be gasified by heating as it is made to flow from the source 2041 to the chamber 2010.

In various embodiments the chamber 2010 can be heated and/or cooled. Also the chamber temperature can be selected to maintain and/or change the phase of an ingredient. For example, a sufficient chamber temperature and/or pressure can be selected to maintain a vaporous compound in the gaseous medium, to avoid condensation in the chamber, and/or to maintain a stable aerosol. In further embodiments, a chamber temperature and/or chamber pressure can be selected to obtain liquid phase and gaseous media coexisting in the chamber. At least a portion of the gaseous media in the chamber can be emanated from the CS system to treat a hand. After emanating a packet, gaseous media can be replenished from liquid phase ingredient in the chamber, and/or from a source. The coexistence can be a thermodynamic equilibrium and/or a kinetic steady state, depending on the application.

An ingredient from source 2041 can be introduced into the chamber through a fluidic passage comprising means such as tubing, a body having fluidic flow channels, and/or others. A quantity and/or flow rate of material from a source into chamber 2010 can be controlled using a valve, a pumping device, an applied pressure, a constriction, and/or various other means.

Aerosolization of a solid and/or liquid ingredient emanating from a source can be effectuated using various means such as evaporation-condensation, piezoelectric and/or other mechanical and/or electromechanical nebulizers, expansion through an orifice and/or nozzle, ultrasonic nebulization, electrospray, spinning disk aerosol generators, and/or other aerosolization means, in single or in combination (for methods of aerosol generation see, for example, Ch. 8, Theoretical Principles and Devices Used to Generate Aerosols for Research, K. H. Leong, in Pharmaceutical Inhalation Aerosol Technology, A. J. Hickey, ed., Marcel Dekker, 2004; Ch. 4.1, Aerosol Particle Generation, R. C. Flagan, in Power Technology Handbook, 3^{rd}. Ed., H. Masuda, K. Higashitani, H. Yoshida, Taylor & Francis, 2006). Although the various methods and/or theories concerning aerosolization disclosed in the Leong reference appear to be consistent with results and data, the claims and/or embodiments herein are not limited by any aerosolization method and/or theory, except where explicitly stated herein.

The chamber 2010 can have various shapes, depending on the application. For example, the chamber can be in the form of a cylinder, a rectangular prism, an ovoid, a surface of revolution, and/or others.

A preselected portion of the gaseous CS medium 2015 in chamber at 2010 can be launched into an ambient 2067 through an opening 2065. In various embodiments the ambient is surrounding atmospheric air. The delivery module 2000 has a launcher 2060 configured to emit the portion of medium from chamber 2010 in the form of a coherent packet 2070 of fluid. The packet of fluid can have preselected form and dimensions and be operable to travel through the ambient atmosphere 2067 to a hand 2510. In a number of embodiments, a packet 2070 can be launched using a pressure differential between the chamber and ambient atmosphere. In some aspects, a packet comprising gaseous CS medium can travel six feet or more in a relatively straight line. The packet can generally maintain a shape as it travels in a direction toward a hand, with relatively little expansion. Furthermore a packet can be launched in a manner operable to maintain expansion during travel within a predetermined maximum amount.

In various embodiments, the CS gaseous medium portion can be launched in the form of a vortex packet (e.g. a vortex ring and/or a spherical vortex). A vortex ring can be generated by imparting an impulse to the portion of the gaseous medium in a manner effective to displace the medium through an opening such as 2065 with respect to FIG. 2A. The impulse and/or displacement can be provided with a pressurizing device and/or displacement means. The pressure and/or displacement can be effectuated using motion of a piston, motion of a diaphragm, a positive pressure of gaseous medium in the chamber, and/or various other means of pressurizing and/or moving the portion of medium to be launched. As the portion of gaseous medium moves through the opening, a boundary layer of the flow separates from the edge of the opening, thereby forming a vortex sheet that rolls up into a vortex ring having the CS gaseous medium. The ring can be generally axisymmetric and can travel away from the opening to a hand.

A vortex ring can generally maintain its shape and size during travel. For example, after launching, the width of a vortex ring transverse to its direction of travel can expand less than 50 percent as it moves through ambient air from the opening to a hand. There are embodiments where the transverse width increases less than 25 percent during travel. Furthermore, there are embodiments where the width of a vortex ring increases less than 15 percent during travel to a hand after it is launched.

While this disclosure does not depend on a vortex packet, or on any theory concerning vortex rings, a vortex ring is particularly useful to send the gaseous medium comprising CS ingredients to a hand. It is believed that the physics of formation and/or motion of a vortex ring can be understood in terms of the Helmholtz requirement that a circulatory fluid motion in unbounded space (e.g. a vortex) forms a loop (see for example Vorticity and Vortex Dynamics, Ch. 6, p. 272, by Wu et al., Springer, 2006). Theoretical equations of fluid motion require that the total circulation in a vortex flow be approximately conserved, and predict that a vortex ring can travel with a relatively constant velocity and can maintain a relatively constant shape and size. In practice it has been found that a vortex ring launching device can launch a vortex ring operable to efficiently transport a selected amount of gaseous medium from a chamber to a distal hand. The size and/or various other aspects of a vortex ring depend on various system parameters such as pressure, an opening size, a launching tube geometry, and/or others that can be preselected. Although theories appear to be consistent with operating data from various embodiments, the claims and/or embodiments herein are not limited by any theory, except where explicitly stated therein.

The media in the chamber can be pressurized by a pressurizing device 2020 with respect to FIG. 2A. In some embodiments the pressurizing device can pump a gaseous medium into the chamber from a pressurized source. For example, the pressurizing device can comprise a source of compressed gas having a control device to control a pressure and/or flow of the source gas into the chamber. In various other aspects, a mechanical compressor can pump a gaseous medium such as air into the chamber. In still further embodiments a pressurizing device can pressurize gaseous medium that is already in the chamber. For example, pressure can be applied to a wall of the chamber with an electromechanical device. In an embodiment shown in FIG. 2B, an electromagnetic actuator such as a loudspeaker comprises a wall of a chamber. An electrical signal can be applied to the loudspeaker to drive a boundary inwardly on chamber 2010, thereby compressing gaseous medium in the chamber. In a further embodiment, shown in FIG. 2C, a piston device can be used for pressurizing chamber 2010.

A delivery module with respect to FIG. 2A, can have a plurality of sources to dispense ingredients into the chamber 2010. For example, in one embodiment with respect to FIG. 2A, there are three sources 2041, 2042, and 2043. An embodiment having different ingredients injected into the chamber by different respective sources can be particularly advantageous in some applications. For example, various ingredients can be difficult to inject together where an association can induce chemical effects. Also, ingredients having inconsistent physical and/or chemical properties can be more conveniently dispensed using different sources. In various embodiments, some ingredients that are immiscible and/or have widely different vaporization/sublimation temperatures are injected into the chamber from different sources. Furthermore, injecting material from distinct sources can be useful to avoid and/or mitigate compositional changes that can arise when extracting vapor above a nonideal liquid and/or solid solution of ingredients. The number of ingredients in a source, the number of sources in a delivery module, a number of CS gaseous media packets launched, and/or a number of ingredients in a launched packet of medium do not limit the scope of the claims.

In further embodiments, a dispenser comprising a plurality of reservoirs allows the various sources to be optimized for particular ingredients and/or delivery rates. For example, the optimal composition for a sanitizing formulation can depend on an temperature, seasonal variations in the prevalence of various different microorganisms, the clinical history of a patient, and/or an expected duty of an employee, healthcare worker, and/or other personnel. It can be seen that gaseous medium comprising various ingredients dispensed from a plurality of ingredient sources can be advantageous.

In further aspects, a hand sanitizing and/or cleansing treatment can comprise a plurality of gaseous media packets having different compositions. In some aspects, various packets having different preselected compositions can be dispensed from the chamber 2010 at different times. For example, at a first time there can be a first composition of gaseous media in the chamber 2010 comprising one or more first preselected amounts of ingredients injected from sources 2041 and 2042. In some embodiments, the first composition can be a sanitizing disinfectant. One or more first gaseous packets comprising the first disinfectant can be sent to a hand. After sending the first packets, a preselected amount of an indicator ingredient from source 2043 can be dispensed into the chamber to form a second gaseous medium composition comprising gaseous indicator. The indicator medium can be operable to interact with microorganisms and provide a detectable indication of sanitizing. The second gaseous indicator medium composition can be sent to a hand in second packets and can indicate whether sanitizing by the first packets is substantially complete. Depending on the indication, further packets of the first sanitizing composition can be sent to the hand.

In some applications an indicator can colorize a hand where there is incomplete sanitizing. The colorizing can be transient (e.g. the signature color can vanish after a predetermined interval of time) or it can be persistent (the color can persist until the sanitizing is complete). In still further embodiments, the chamber of the delivery module can receive a third composition from a third source and send the third composition to a hand. The third composition can be to decolorize an indicator.

In various applications, a system can have sensors to detect an indicator response. There can be an indicator that is imperceptible to human senses. For example, there can be an indicator that is detectable using short and/or long wave electromagnetic radiation beyond the spectrum of visible light (e.g. ultraviolet, infrared, radiofrequency, microwave frequency wavelengths, etc.). A hand can be illuminated with visible and/or invisible radiation to induce a detectable signal from any areas of a hand where sanitizing is complete and/or incomplete.

There are embodiments where a packet of gaseous medium can comprise a hand cleansing and/or sanitizing formulation and an indicator in combination. The indicator can be a chromophore that is operable to develop a color on a hand. For example, in some applications, a chromophore can manifest a color in bonding with a protein of bioactive contamination. In some aspects, an indicator signal can be developed by rubbing. For example, there are embodiments where hands can be rubbed to induce colorizing. The colorizing can disclose areas where there is insufficient cleansing and/or sanitizing. Responsive to the colorizing, a user can effectuate further sanitizing by inserting a hand into the system. The system can detect the presence of the hand, colorizing, and/or another indicator signal and can responsively send further CS packets to the hand.

It should be understood that the injection of one or more materials from various sources in a chamber, and/or emitting the gaseous medium in packets, can be sequenced in various different ways, depending on the application. Furthermore the chamber can be evacuated and/or purged before injecting various ingredients into a chamber to be emanated in packets of gaseous media sent to a hand. For example, after forming and emanating a gaseous medium in one or more sanitizing packets, a chamber can be cleaned by purging the chamber with clean air from a clean air source. The clean air can sweep contents of a chamber into an exhaust line, and/or the contents can be evacuated through an opening 2065. In some embodiments, a launching device can be configured to vent the chamber contents diffusely in a local area proximate to the chamber, without sending packets. Furthermore, some embodiments have a separate vent opening and flow diverting means operable to selectively purge a chamber to an exhaust line, or to direct the chamber contents to the launcher. However, in a number of applications, purging and/or venting is unnecessary and means for purging and/or venting are not provided.

There are embodiments where a launching device 2060 is operable to send a packet of gaseous medium in a selectable direction. For example, in some embodiments with respect to FIG. 2A, a launching device can be turned upward 2080 and/or downward 2082 to direct a packet along a trajectory to a hand in a respective position above or below the hand position 1510 shown in FIG. 2A. A launching device can also be turned in other directions such as a leftward or rightward direction to send a packet along various other trajectories. Furthermore, there are embodiments having a static steerable launching device. In some embodiments, a launching device can launch a packet of the medium in a selected direction by emitting preselected amounts of gaseous medium through two or more apertures in an array. The apertures can be relatively close to one another and can have round, elliptical, and/or other shapes, depending on the application. The launching device can emit the preselected amounts of gaseous medium from selected apertures of an array starting in a preselected sequence (e.g. having preselected phases) with respect to one another. The aperture selection, preselected amounts, and relative phase can cooperatively launch a packet of gaseous medium in the form of a vortex ring having a predetermined direction and/or speed. Although the operation of such a phase steerable emitter does not depend on any theory, it is believed that constructive and/or destructive interference of the respective vorticity fields emanating from the selected apertures can effectuate fluidic coherence in a vortex motion.

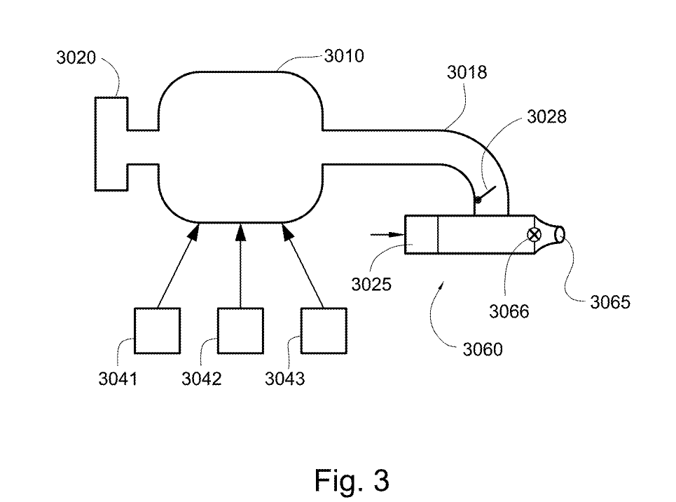

Various embodiments with respect to FIG. 3 can comprise a pressurizing device 3020, a chamber 3010, one or more sources such as 3041, 3042, 3043, and can have a modular launching device 3060 comprising an opening means 3065 and second pressurizing device 3025 for the launching device. In some aspects, the injecting of ingredients from the one or more sources (e.g. 3041, 3042, 3043 and/or others) is operable to pressurize the chamber 3010 sufficiently to power launching. Accordingly, some of these aspects have no chamber pressuring device 3020.

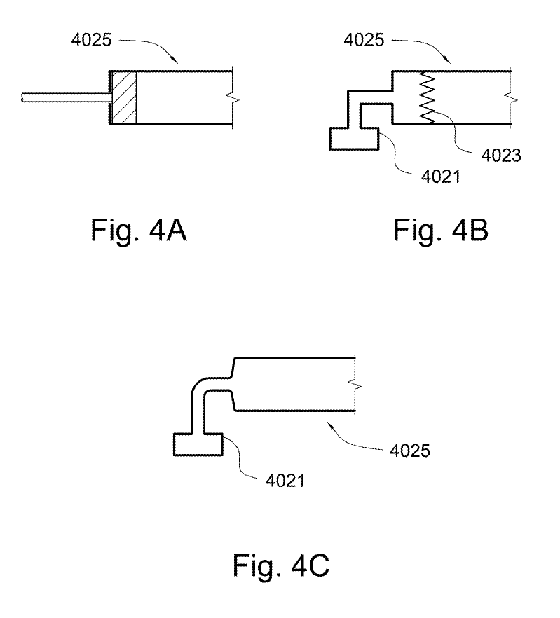

A launching device 3060 according to FIG. 3 can have a dedicated pressuring device 3025, and an opening 3065 for launching packets of gaseous media. The launching device can be coupled with fluid passage 3018 to chamber 3010 and there can be a flow control device 3028 to control fluid flow between the chamber 3010 and launching device 3060. A pressurizing device can be a movable wall, a source of compressed gas, a piston, and/or others. For example, the dedicated pressurizing device can be a positive displacement means such as a piston shown with respect to the simplified diagram FIG. 4A, a flexible diaphragm and/or bellows 4023 movably coupled to a pressure and/or vacuum source 4021 with respect to the simplified diagram FIG. 4B or, as represented in the simplified diagram of FIG. 4C, a pressurized gas source 4021 coupled to the launching device 4025. However these embodiments, and the number and/or configuration of various launching and/or pressurizing devices do not limit the scope of the claims, except where expressly stated therein.

In an embodiment with respect to FIG. 3, a composition of gaseous media is formed by injecting ingredients from the one or more sources into the chamber 3010, the flow regulating device 3028 and/or a valve 3066 can cooperatively gate a preselected portion of gaseous media from chamber 3010 into launching device 3060 through fluidic communication means 3018. The preselected portion of gaseous medium in the launching device 3060 can be launched from the opening 3065. There are launching device embodiments that can selectively launch one or more packets in a directional manner as disclosed above with respect to FIG. 2A. For example, in some embodiments a launching device as a whole, and/or a portion of the launching device comprising opening 3065 can adjustably select a direction of travel, an amount of gaseous medium, a speed and/or other attributes of a discrete gaseous medium packet and its trajectory.

With respect to FIG. 2A, various steps and/or operations such as the selection and injection of ingredients from sources 2041, 2042, 2043, etc. into a chamber 2010, pressurization of the chamber using pressurizing means 2020, detecting the position of a hand 2510, selecting a trajectory for a packet to reach a hand, and/or others can be effectuated with one or more control circuits. Furthermore, in some embodiments control and sequencing can be effectuated using instructions and or data (also referred to as software) stored in machine readable media 2093 operable to be performed by a processor 2092 of the hand cleansing and/or sanitizing system. For example, in one embodiment, one or more sensing devices such a first sensor device 2034 and/or a second sensor device 2036 can detect a hand 2510. Active and/or passive sensors can be used, depending on the application. For example, a sensor can be based on sending a pulse of ultrasound to intercept a hand, and can sense reflected ultrasound. In another aspect, hand sensing can be performed using an array of infrared heat sensitive charge coupled devices (CCD).

In various embodiments, after a user hand is placed in an operable position, a system can clean and/or sanitize a hand without human interaction. In further embodiments, a system can send an auditory, visual, electronic, and/or other signal operable to communicate that a step of a cleansing/sanitizing process is complete. In some embodiments, an auditory and/or visual signal can signal a user that a hand or other object has been sufficiently cleaned and/or sanitized. Furthermore, a signal can be sent to an automated control and/or monitoring system. For example, a signal can be sent to a control system for a biohazard containment facility, a cleanroom, and/or other contamination control systems. The signal can be based on completion of a series of process steps, a sensor responsive to a contaminant and/or microorganism, and/or others.

Using one or more software hand sensing modules comprising instructions in media 2093 for processing sensor data, a processor 2092 can receive data from the CCDs and determine the presence, position and/or state of a hand. In some embodiments, a module can determine whether cleansing/sanitizing has been effectuated based on sending a packet comprising an indicator ingredient to a hand. Attributes of the indicator can be responsive to the presence of various substances and/or biological vectors. For example, an indicator can be operable to fluoresce at predetermined wavelengths depending on an amount and/or composition of a material on a hand. In some embodiments, a source of infrared, visible, and/or ultraviolet illumination 2034 can be useful to stimulate the fluorescence. The illuminating wavelength, duration of illumination, and/or intensity can be selected using a software module performed by the processor. In some embodiments a software module can select one or more wavelengths of illumination operable to stimulate respective signals corresponding to different substances and/or biological vectors. In one embodiment the illumination source 2034 is a light emitting diode having an emission intensity and/or wavelength that can be effectuated using electrical signals applied with the software module. Furthermore, there can be software modules operable to selectively effectuate a quantity and/or composition of gaseous medium in a packet, launching parameters for sending a packet, sending a number of packets, a time interval between launching the various packets, and/or various further parameters that can optimize hand cleansing and/or sanitizing.

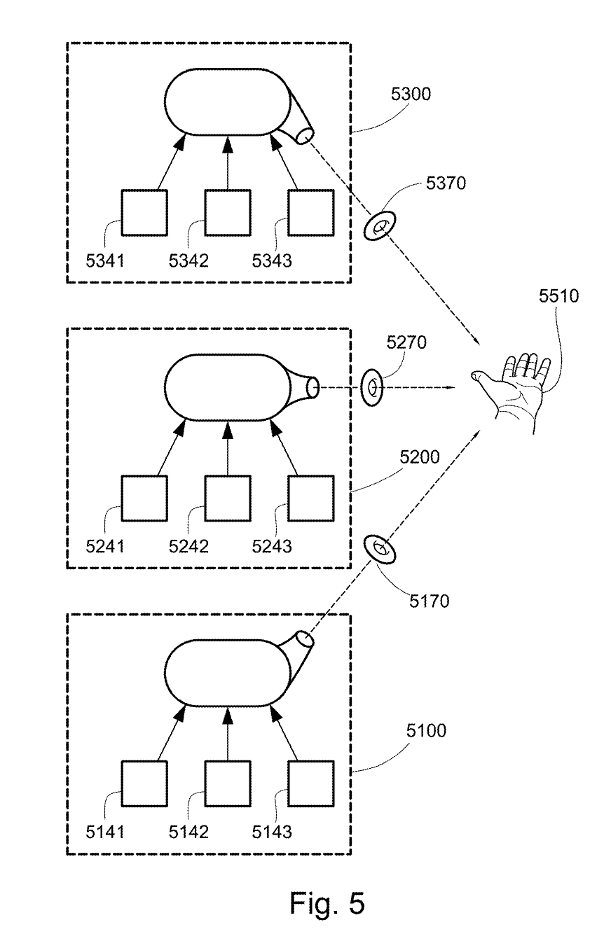

Still further embodiments can be understood with respect to a simplified diagram shown in FIG. 5. Various embodiments comprise a plurality of delivery modules such as 5100, 5200, 5300. Each of the respective delivery modules has various respective ingredient sources, a launcher, and can have sensors, control circuits, a processor and software modules, and/or various other elements as disclosed above with respect to FIGS. 1A-1C, 2, 3A-3C. The various delivery modules can be placed in various positions and/or in various orientations and can be configured to launch packets having various different compositions.

Furthermore, the various delivery modules can have limited purpose launchers. A limited purpose launcher can be optimized to launch packets having certain characteristics. For example, there can be a first launcher operable to launch packets in a first range of sizes, a first range of speeds, and/or a first range of directions. Furthermore, the first launcher can be compatible with gaseous media having first physical and chemical properties. A second launcher can launch packets operable to launch a different range of sizes, speeds, and/or directions, and can be compatible with physical and or chemical gaseous medium properties that are inoperable and/or incompatible with the first launcher. Also, the various delivery modules in a system can be coupled to different types of sensors, different numbers of sensors, and/or can have no sensors, depending on the application. It will be understood that a system having a plurality of limited purpose delivery modules can be particularly useful where a universal module design would be more costly and/or impractical.

In some embodiments a system comprising a plurality of delivery modules relative to FIG. 5 can have at least one master control module to effectuate cooperative dispensing among at least some of the control modules for the various delivery modules. In some embodiments, there can be common components that perform a function for one or more modules. For example, there are embodiments where a microprocessor can effectuate a first control module comprising first data and instructions operable to launch a packet of gaseous media from a first delivery module, and can effectuate a second control module comprising different data and instructions to launch a packet of a different gaseous medium from a second delivery module. Furthermore, there are embodiments where some modules can be used in different delivery modules (e.g. a component such as a pressurizing device, a sensor, an ingredient source, etc.) can be shared among two or more delivery modules). For example, a software module for launching a packet can be operable to launch packets from various launching devices at the same and/or at various different times, and/or in various ways, depending on the embodiment.

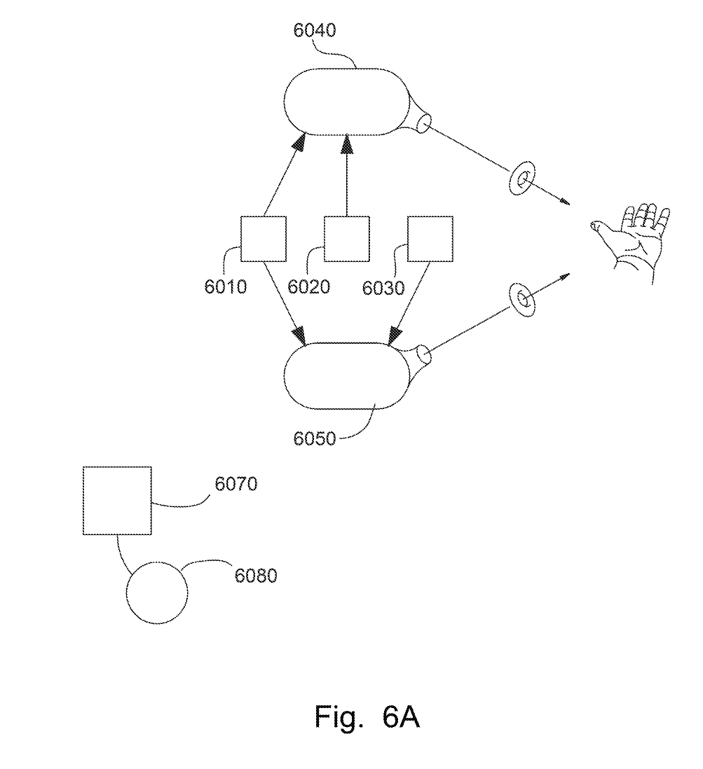

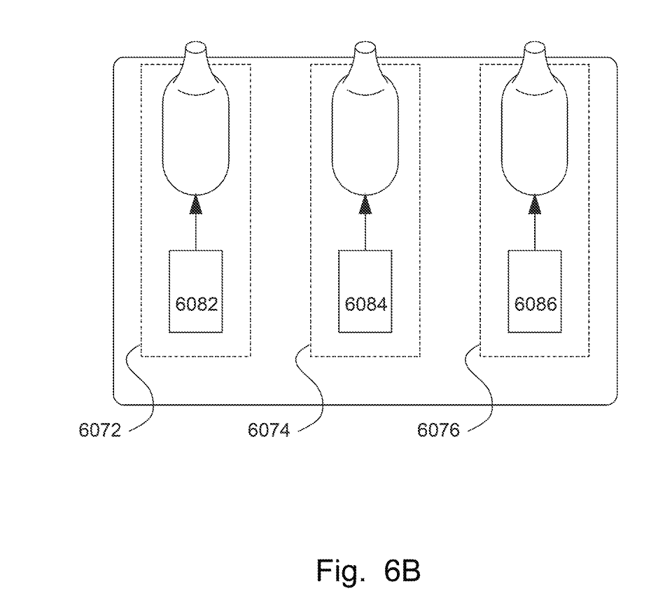

Although launchers 5100, 5200 and 5300 are shown to have distinct ingredient sources 5141, 5142, 5143, 5241, 5242, 5243, and 5341, 5342, 5343, one ingredient source can be configured to supply one or more ingredients to at least two modules, as has been pointed out above. For example, with respect to the simplified diagram in FIG. 6A, an ingredient source 6010 is operable to admix ingredients into the chambers, 6040 and/or 6050, by way of fluid passages to different delivery modules. Furthermore, the injecting can be controlled using a common software control module comprising instructions in media 6080 that are operable to be performed by a processor 6080. An embodiment having two delivery modules can be useful for cleansing and/or sanitizing two hands at once.

There are embodiments where a plurality of delivery modules can be housed in one hardware module. For example, with respect to FIG. 6B, three delivery modules 6072, 6074, and 6076 are housed in one unit. Although each of delivery modules is shown to have distinct components such as respective ingredient sources 6082, 6084, and 6086, a single ingredient source, a single control processor (as shown with respect to FIG. 6A), and/or various other components of a system can support one or more delivery modules, depending on the application.

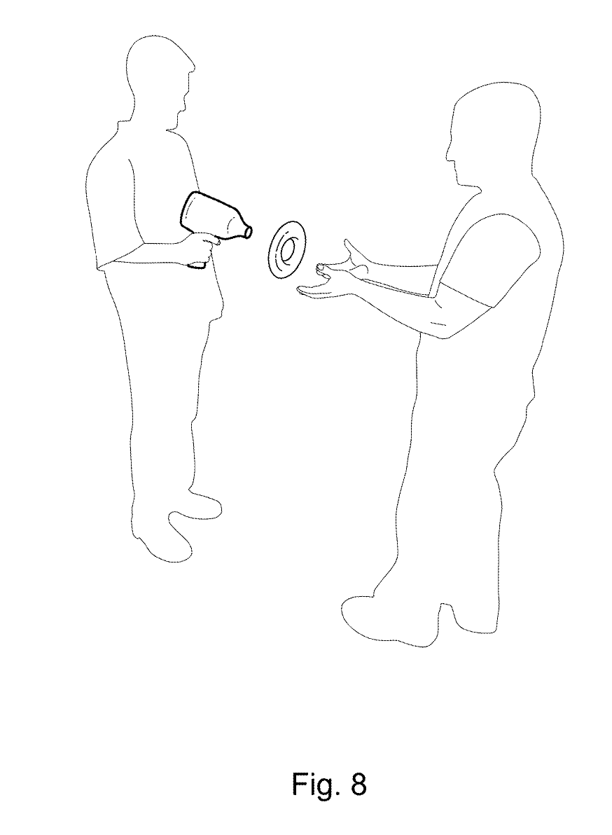

Although the various embodiments for cleansing and/or sanitizing hands have been disclosed, it will be appreciated that the various embodiments and various further embodiments can be useful for further cleansing and/or sanitizing applications. For example, the disclosed embodiments can be useful for cleansing and/or sanitizing feet, arms, and/or various other body parts. Furthermore there are embodiments that can be useful for cleansing and/or sanitizing various objects such as a countertop, a cosmetic tool, a surgical tool, and/or others. For example, FIG. 8 shows a portable and/or mobile embodiment of a hand cleansing and/or sanitizing apparatus operable to send a packet of gaseous medium that is useful to clean and/or sanitize hands. However it will be understood that an embodiment can also be used to clean and/or sanitize a countertop, a doorknob, and instrument and/or other things. Furthermore, cleansing/sanitizing devices, processes, and/or systems according to the instant disclosure can be useful to automate various systems. For example, an embodiment can be useful in a robotic device to automatically clean and/or sanitize countertops, a robotic surgery system, remote telepresence healthcare facilities, and others.

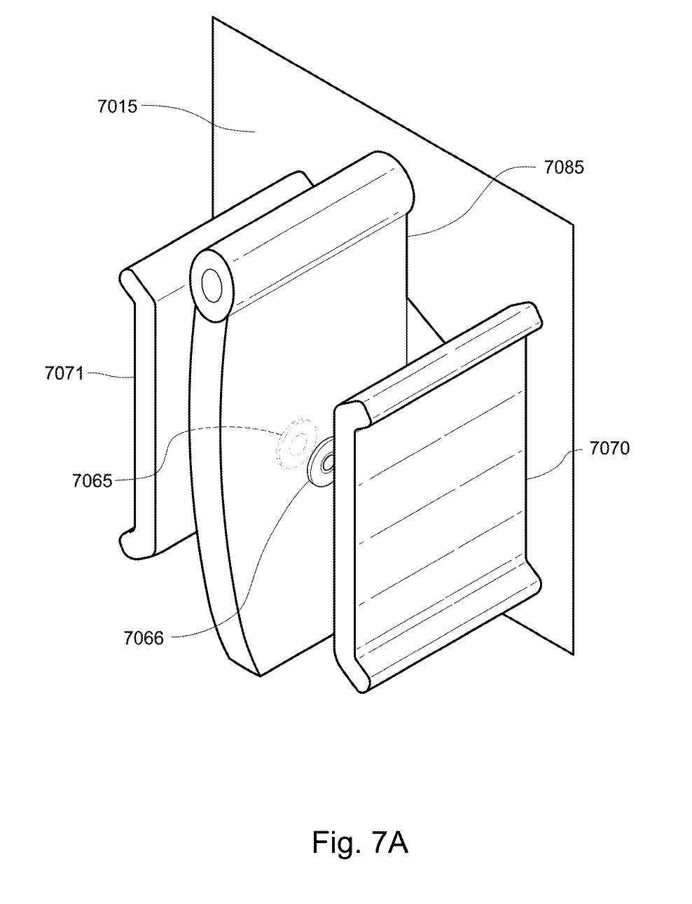

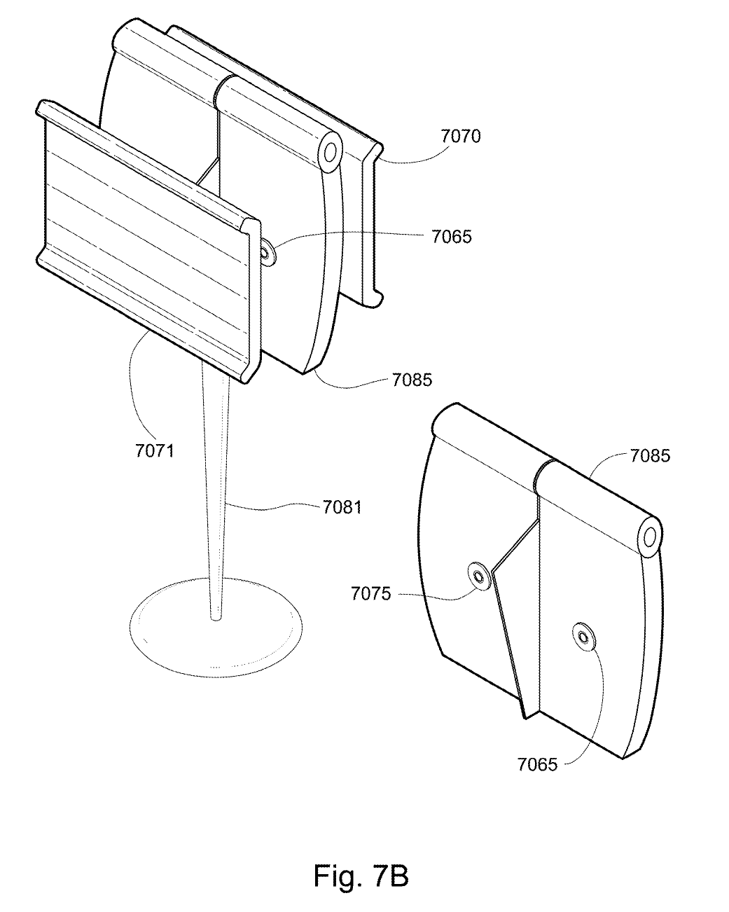

Some further embodiments are shown with respect to FIGS. 7A-7B. FIG. 7A shows a hand cleansing/sanitizing assembly that can be mounted on a wall 7015. There is a housing 7085 comprising leftward and rightward directed delivery modules having one common control module and common ingredient sources. Furthermore, in this embodiment, the leftward and rightward delivery modules share a common gaseous medium chamber. However the leftward and rightward delivery modules comprise separate rightward and leftward launchers having respective openings 7065 and 7066. The control module and ingredient sources can inject ingredients into the common chamber that is operable to supply CS gaseous media to the launcher of each delivery module. There is a sensor (not shown) operable to detect a hand in the space between housing 7085 and baffle plate 7071, and another sensor operable to detect a hand in the space between housing 7085 and baffle plate 7070. A vortex ring having a gaseous sanitizing formulation can be directed from the left launcher through the left outlet 7065, and/or from the right launcher through the right outlet 7066, to a hand detected in the respective right and/or left space. The baffle plates 7071 and 7070 are to limit effusion of any excess media into the environment of a room.

FIG. 7B shows a unit having four dispensers configured with a floor stand. In additional to baffle plates 7071 and 7070, there are partitions separating two background dispenser openings (only one opening 7075 is shown in the FIG. 7B) from two foreground dispenser openings 7065, 7085. The unit can concurrently process a right and left hand of one person facing the unit from the foreground, and a right and a left hand of a second person behind the unit facing forward.

In the foregoing specification, various aspects are described with reference to specific embodiments, but those skilled in the art will recognize that further aspects are not limited thereto. Various features and aspects described above may be used individually or jointly. Other aspects of the invention, including alternatives, modifications, permutations and equivalents of the embodiments described herein, will be apparent to those skilled in the art from consideration of the specification, study of the drawings, and practice of the various aspects. Further, various aspects can be utilized in any number of environments and applications beyond those described herein without departing from the broader spirit and scope of the description. The written description and accompanying drawings are, accordingly, to be regarded as illustrative rather than restrictive.

The embodiments and preferred features described above should be considered exemplary, with the invention being defined by the appended claims, which therefore include all such alternatives, modifications, permutations and equivalents as fall within the true spirit and scope of the present disclosures.

1-21. (canceled)

22. A hand cleansing-sanitizing system comprising:
a. a first vortex ring delivery module that is leftward directed;
b. a second vortex ring delivery module that is rightward directed;
c. a control module;
d. an ingredient source;
e. a gaseous medium chamber;
and wherein said control module, said ingredient source and said gaseous medium chamber are commonly shared by said first vortex ring delivery module and said second vortex ring delivery module such that an ingredient injected from said ingredient source via said control module into said gaseous medium chamber is delivered by at least one of said first vortex ring delivery module in a left vortex ring to a left hand and of said second vortex ring delivery module in a right vortex ring to a right hand.

23. The hand cleansing-sanitizing system of claim 22, further comprising at least one of a baffle plate in a space to one of said first vortex ring delivery module and said second vortex ring delivery module such that effusion of any excess of said ingredient into an environment is limited while one of said left vortex ring and said right vortex ring is delivered towards a hand inserted in said space.

24. The hand cleansing-sanitizing system of claim 23, further comprising a sensor to detect a hand in said space.

25. The hand cleansing-sanitizing system of claim 22 being wall mounted.

26. The hand cleansing-sanitizing system of claim 22, further comprising a floor stand.

27. The hand cleansing-sanitizing system of claim 26, further comprising:
a. two foreground of said first vortex ring delivery module and said second vortex ring delivery module;
b. two background of said first vortex ring delivery module and said second vortex ring delivery module; and
b. two partitions each of which separating one of said foreground delivery modules from one of said background delivery modules such that said hand cleansing-sanitizing system is capable of concurrently processing a right and a left hand of one person facing said hand cleansing-sanitizing system from the foreground, and a right and a left hand of a second person behind said hand cleansing-sanitizing system facing forward.

28. A method for hand cleansing-sanitizing comprising the steps of:
a. placing a hand in front of a vortex ring delivery module;
b. injecting a cleansing-sanitizing ingredient into a gaseous medium chamber thereby creating a gaseous mixture containing said injected cleansing-sanitizing ingredient;
c. delivering said gaseous mixture in a vortex ring from said vortex ring delivery module towards said hand in a fashion such that said vortex ring dissipates in a brief swirling over said hand thereby reaching various areas including crevices on a front and of a back of said hand and applying said cleansing-sanitizing ingredient thereon.

29. The method of claim 28, wherein said hand is a single hand.

30. The method of claim 29, wherein said vortex ring is a single vortex ring.

31. The method of claim 28, wherein multiple of said vortex ring are delivered during step c).

32. The method of claim 31, wherein said multiple vortex rings are delivered at an interval.

33. The method of claim 28, further comprising following step b) and prior to step c) the step of injecting an indicator ingredient to said gaseous medium, said indicator ingredient being operable to disclose a sufficiency of cleansing-sanitizing at said various areas.

34. The method of claim 33, wherein said indicator ingredient is chromophore that is manifesting a color in bonding with a protein of bioactive contamination.

35. The method of claim 34, further comprising following step c) the steps of injecting a second indicator ingredient to said gaseous medium and delivering a second vortex ring containing said second indicator ingredient, whereby said second indicator ingredient discolors said first indicator ingredient.
